(19) United States
(12) Patent Application Publication
(10) Pub. No.: US 2013/0211246 A1
(43) Pub. Date: Aug. 15, 2013

(54) METHODS AND DEVICES FOR GASTROINTESTINAL SURGICAL PROCEDURES USING NEAR INFRARED (nIR) IMAGING TECHNIQUES

(71) Applicant: Vinod PARASHER, Washington, DC (US)

(72) Inventor: Vinod PARASHER, Washington, DC (US)

(21) Appl. No.: 13/722,220

(22) Filed: Dec. 20, 2012

Related U.S. Application Data
(60) Provisional application No. 61/580,470, filed on Dec. 27, 2011.

(51) Int. Cl. A61B 1/273; A61B 1/00; A61M 5/00; A61B 1/06; A61B 1/018; A61M 25/09; A61B 5/00; A61B 1/04

(52) U.S. Cl. CPC A61B 1/273; A61B 5/0075; A61B 5/0086; A61B 1/00186; A61B 1/042; A61B 1/00045; A61B 1/0638; A61B 1/0676; A61B 1/00096; A61B 1/018; A61M 25/09; A61M 5/007
USPC 600/431; 600/473

(57) ABSTRACT

Described herein are methods and devices for performing gastrointestinal surgical procedures using near infrared (nIR) imaging techniques. Described herein are imaging systems, endoscopes, and methods making use of near infrared (nIR) imaging techniques. The imaging systems, endoscopes, and methods can be used, for example, in endoscopic retrograde cholangiopancreatography (ERCP) for visualization of the intraduodenal portion of the bile duct, and in procedures to visualize and to direct treatment of bleeding ulcers, gastrointestinal bleeding, and tumors, for example, a pancreatic mass.

### CROSS-REFERENCE TO RELATED APPLICATION

This application claims priority to U.S. Provisional Application for Patent Ser. No. 61/580,470 filed Dec. 27, 2011, the disclosure of which is incorporated herein by reference in its entirety.

### FIELD OF THE DISCLOSURE

Described herein are methods and devices for performing gastrointestinal surgical procedures using near infrared (nIR) imaging techniques. Described herein are imaging systems, endoscopes, and methods making use of near infrared (nIR) imaging techniques. The imaging systems, endoscopes, and methods can be used, for example, in endoscopic retrograde cholangiopancreatography (ERCP) for visualization of the intraduodenal portion of the bile duct, and in procedures to visualize and to direct treatment of bleeding ulcers, gastrointestinal bleeding, and tumors, for example, a pancreatic mass.

### BACKGROUND

ERCP is an endoscopic technique that involves placement of a viewing instrument, for example, an endoscope or a duodenoscope, within the duodenum. ERCP is an alternative to invasive surgery for the identification and/or treatment of obstructions and abnormalities of the biliary and pancreatic ducts. It is possible to pass additional medical devices through the endoscope for treatment and diagnostic purposes, for example, a catheter, or for navigation purposes, for example, a guide wire.

A physician can perform ERCP by inserting and guiding an endoscope through a patient's alimentary canal, esophagus, stomach, and duodenum. The physician can stop advancing the endoscope upon reaching the area of the duodenum where the ducts of the biliary tree and the pancreas open into the duodenum. This opening, commonly referred to the ampulla of Vater or papilla of Vater, is a small mound of tissue which resembles a nipple in appearance.

After guiding the endoscope to the ampulla of Vater, a catheter, for example a biliary catheter, can be inserted through the endoscope so that the distal end of the biliary catheter emerges from the distal end of the endoscope. The biliary catheter can be used for cannulation of the ampulla in order to access additional anatomical structures, for example, the intraduodenal portion of the bile duct, the common bile duct, and the pancreatic bile duct. Cannulation of the ampulla can be achieved by probing the duodenum and ampulla tissue with the endoscope and, if necessary, a guide wire. However, despite best efforts, a physician can be unsuccessful in cannulation. Additionally, excessive probing of the tissue can lead to inflammation and patient discomfort. Although an endoscope can be equipped with a visualization apparatus, for example a direct light visualization apparatus, this does not eliminate or greatly reduce the difficulty in cannulation of the ampulla.

### SUMMARY OF THE DISCLOSURE

Described herein are methods and techniques for performing ERCP procedures that overcome these difficulties. The methods described herein can make use of nIR imaging techniques. For example, methods described herein include ERCP using an endoscope capable of nIR emission and detection, and fluorescent dyes capable of emitting nIR light upon excitation. Methods and devices described herein also include esophagogastroduodenoscopy (EGD) for diagnosis and treatment of bleeding ulcers and other gastrointestinal bleeding. The nIR imaging techniques described herein can also be used to visualize and direct biopsy and treatment of tumors, for example, including tumors near the epithelial surface and pancreatic masses near the pancreatic duct.

An imaging system for an endoscope as described herein can comprise the following: a light source capable of emitting near-infrared light; an objective lens; an optical filter; an imaging sensor; and an image display unit. The light source can emit nIR light having a wavelength of about 800 nm. The optical filter can be, for example, a band-pass filter centered at about 830 nm or a long-pass filter excluding light below about 810 to 820 nm. Alternatively, the light source can also emit visible light in addition to nIR light. The imaging sensor can be a camera, for example, a solid state nIR sensitive camera. The image display unit can be a monitor.

An endoscope as described herein can comprise a distal portion for insertion into a patient; a proximal portion which is external to the patient; and an imaging system having a light source capable of emitting near-infrared light; an objective lens; an optical filter; and an imaging sensor. The light source and objective lens can be positioned at a distal end of the endoscope. The filter and imaging sensor can be positioned at a proximal end of the endoscope. The endoscope can further comprise at least one lumen for insertion of additional medical devices, for example, catheters and guide wires.

The light source can emit nIR light having a wavelength of about 800 nm. The optical filter can be, for example, a band-pass filter centered at about 830 nm or a long-pass filter excluding light below about 810 to 820 nm. Alternatively, the light source can also emit visible light in addition to nIR light. The imaging sensor can be a camera, for example, a solid state nIR sensitive camera.

An ERCP method as described herein can comprise the following steps: injecting a patient with a dye which emits or fluoresces near-infrared light upon excitation and which is absorbed into biliary epithelium; positioning, in the patient's duodenum, an endoscope capable of emitting and detecting near-infrared light; emitting near-infrared light from the endoscope; detecting near-infrared light emitted or fluoresced by the dye to provide an image for guidance of the endoscope for cannulation of the intraduodenal portion of the bile duct; and cannulating the intraduodenal portion of the bile duct.

### BRIEF DESCRIPTION OF THE DRAWINGS

FIG. 1 is a perspective view of an endoscope in accordance with the present disclosure.

FIGS. 2A to 2C are perspective views of a distal end of an endoscope of the present disclosure.

FIG. 3 is a diagram of the anatomy of the ampulla of Vater.

FIG. 4 is a photograph of an endoscopic view of the ampulla which is visualized without nIR light.

FIG. 5 is a diagram of the portion of the bile duct to be visualized using nIR light.

### DETAILED DESCRIPTION

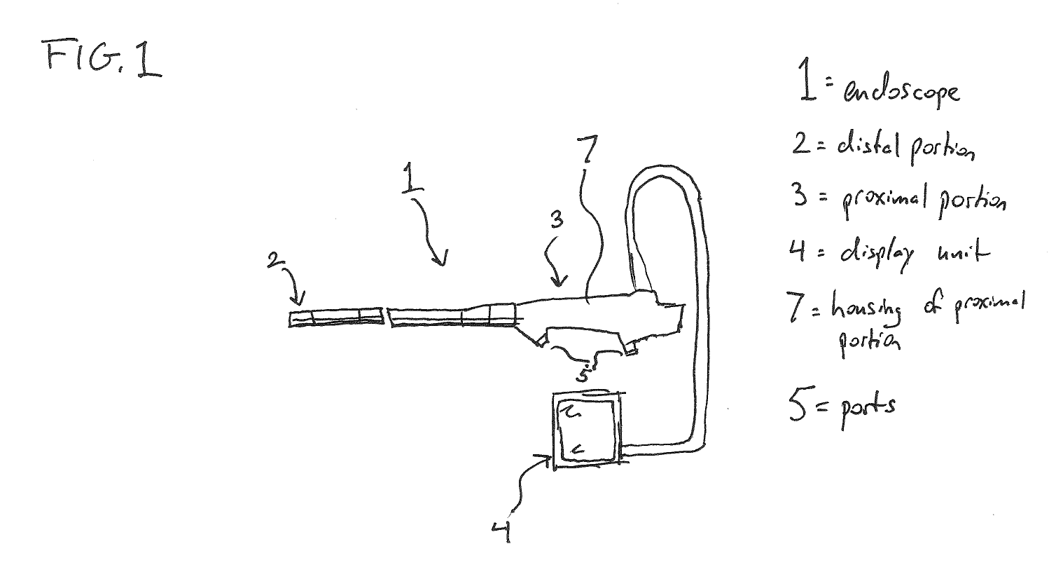

As shown in FIG. 1, an endoscope 1 as described herein can have a distal portion 2 for insertion into a patient; a proximal portion 3 which is external to the patient; and an imaging system which is capable of emitting nIR light and detecting fluorescence emission from dyes after excitation by nIR light. For example, when ICG is used as a dye, the endoscope can be a fiber optic endoscope which includes an imaging system having a light source for nIR emission at 800 nm (the excitatory wavelength for ICG), and objective lens, and an optical filter to allow only 830 nm wavelength light (the fluorescence wavelength of ICG after excitation) to enter an imaging sensor, for example, a camera. The imaging system can also include a filter. This imaging system can provide fluorescent imaging of ICG after excitation with nIR. The endoscope can also have an image display unit 4. The endoscope can be provided with one or more ports 5 located, for example, in the proximal portion, for use with medical devices, including, but not limited to, guide wires, cannulae, and catheters.

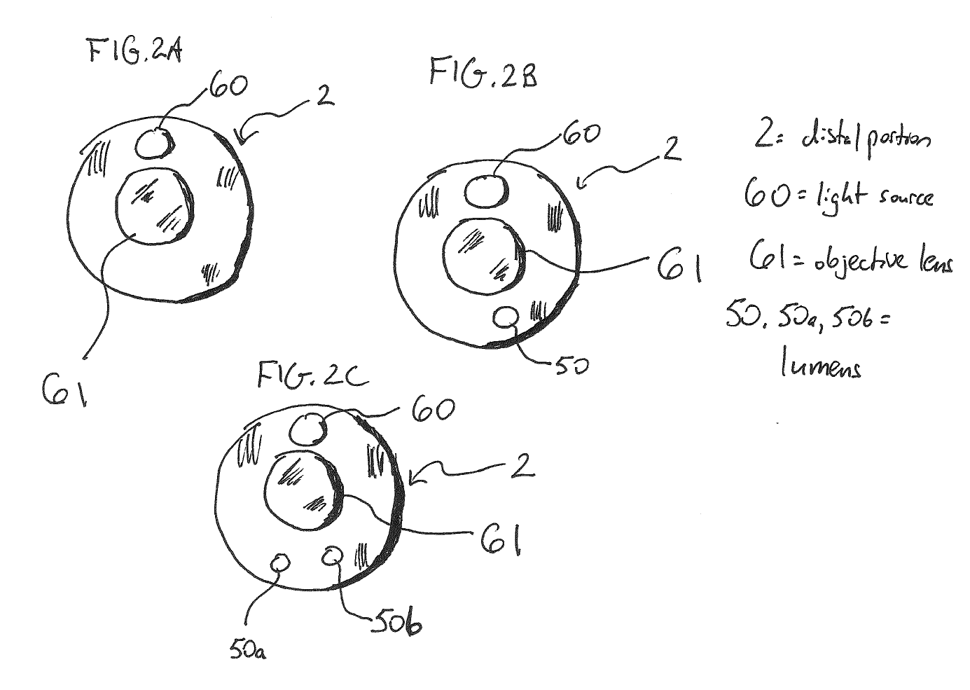

As shown in FIGS. 2A-2C, the distal portion 2 of the endoscope can include a light source 60 and an objective lens 61. The endoscope as described herein can include at least one lumen 50 through which an additional medical device can be inserted into the patient (FIG. 2B.) For example, the endoscope can include a lumen for insertion of a catheter or a guide wire. The endoscope can also have multiple lumens 50a, 50b (FIG. 2C.)

The endoscope as described herein can be adapted for use with a particular gastrointestinal procedure. For example, the endoscope can be adapted for use in ERCP or EGD.

Suitable optical filters for the imaging system used with ICG include a band-pass filter centered at 830 nm or a long-pass filter excluding light below 810 to 820 nm. The filter used in the imaging system can be adjusted based on the type of dye used during ERCP.

The filter and imaging sensor can be positioned at the proximal end, i.e., the end which is located externally of the patient, of the endoscope. For example, the filter and imaging sensor can be positioned in housing 7 of the proximal section of the endoscope. Positioning at the proximal end may be advantageous if the nIR sensitivity of solid state cameras used at the distal end of the endoscope is indeterminate and insufficient. Additionally, manipulation of the captured image with a band-pass or long-pass filter, for example, to exclude the excitatory nIR at 800 nm and to allow imaging at 830 nm when using ICG, can be easier to accomplish at the proximal end of the endoscope.

Imaging sensors suitable for use in the systems and methods described herein may include solid state nIR sensitive cameras, for example, CMOS board cameras. The optical path between the proximal end of the endoscope and the imaging sensor can be designed to permit the insertion or removal of light filters.

Referring to FIGS. 2A-2C, the endoscope can include an objective lens 61 at the distal end thereof. The image captured by the objective lens, for example, fluorescence emission following excitation of the dye with nIR light, can be transmitted via optical fibers to the proximal end of the endoscope and through the filter for projection into the imaging sensor.

The light source 60 for emitting nIR light can be a light box. The light box can be positioned at the distal end, i.e., end which is located internally of the patient, of the endoscope. Light boxes suitable for use in the systems and methods described herein can be capable of emitting only nIR light or can be capable of emitting both nIR light and visible light. For example, in a light box capable of emitting both nIR light and visible light, light transmissive optical fibers can used to transmit either nIR or visible light, as selected by the operator, from the light box. The endoscope can also have a separate light source for emitting visual light (not shown.)

The shape, size, and positioning of the objective lens, light source, and optional lumen(s) at the distal end of the endoscope is not particularly limited. The objective lens, light source, and optional lumen(s) can be configured as needed for a particular procedure as understood by a person of ordinary skill in the art.

Additional visualization options can be obtained by removing the filters from the imaging system. Removal of the filters can provide nIR visualization of vasculature at 800 nm, for example, providing images of vasculature generated by reflection of the 800 nm light more greatly absorbed by hemoglobin containing blood vessels than other tissues. For an endoscope having a light box that emits both nIR and visible light source, removal of the filters in the visible light mode can allow operation with visible light.

As shown in FIG. 1, the image display unit 4 can be a monitor connected to the endoscope. However, the form and positioning of the image display unit is not particularly limited. For example, in an alternative configuration, the image display unit can be integrated with the proximal portion of the endoscope, for example, as a LCD screen.

A computer (not shown) can be used to control the pulsing and intensity of the nIR light source, and the visible light source, if present, for image generation. The computer can also be used to synchronize gating of the camera and pulsing of the light source(s). For imaging systems having a light box capable of emitting both nIR light and visible light, the nIR and visible image can be overlaid via image processing.

The methods described herein include ERCP procedures in which the intraduodenal portion of the bile duct is visualized by nIR light. In order to permit visualization by nIR light, it is necessary for the biliary epithelium to absorb a dye which can emit nIR light. Such dyes can be introduced to the biliary epithelium, for example, by intravenous injection of the dyes into the patient.

Suitable dyes for use in the methods described herein include indocyanine green (ICG). ICG has a maximum absorption (excitation) wavelength of 800 nm. ICG emits light with a peak wavelength of approximately 830 nm when illuminated with nIR light (fluorescence wavelength of ICG after excitation). Other dyes which absorb and emit fluorescence within the nIR spectrum are also suitable for use in the methods described herein.

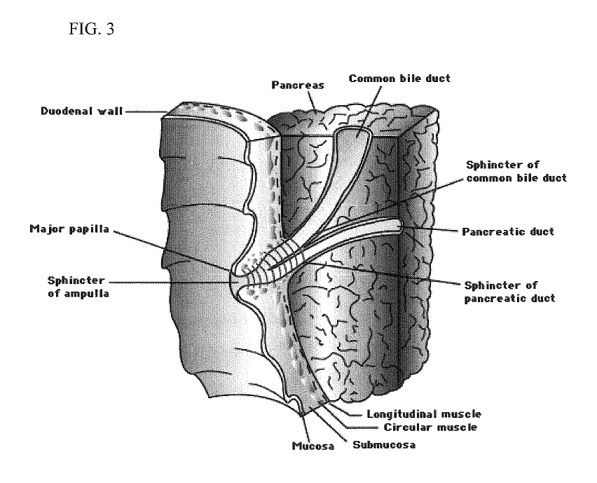

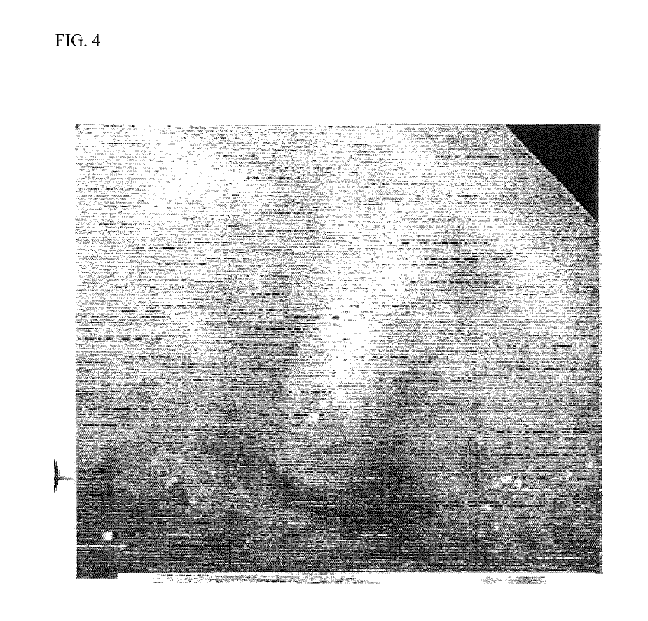

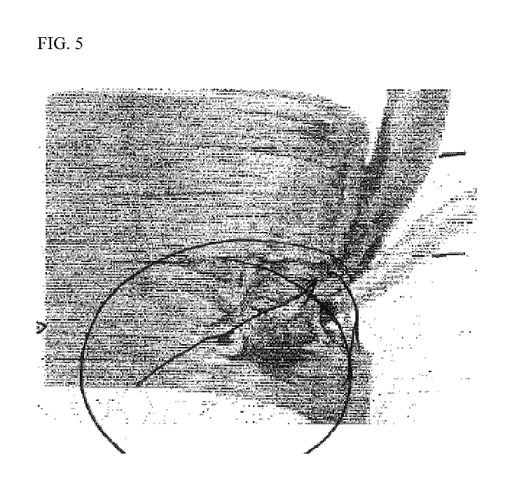

FIGS. 3-5 show anatomical areas which can be targeted by the methods described herein. The methods can include positioning the endoscope in the patient's duodenum. Emitting near-infrared light from the endoscope and detecting near-infrared light emitted by the dye can be used to provide an image of the duodenum for guidance of the endoscope. The image can be used to aid in cannulation of the intraduodenal portion of the bile duct for the ERC procedure.

The methods and devices described herein can be used in other gastrointestinal procedures, for example, esophagogastroduodenoscopy (EGD) procedures for diagnosis and treatment of bleeding ulcers and other gastrointestinal bleeding. The endoscope described herein can be adapted for use in EGD. An EGD method as described herein can comprise the following steps: injecting a patient with a dye which emits or fluoresces near-infrared light upon excitation and which is absorbed into the epithelium; positioning, in the patient's esophagus, stomach, and/or duodenum, an endoscope capable of emitting and detecting near-infrared light; emitting near-infrared light from the endoscope; detecting near-infrared light emitted or fluoresced by the dye to provide an image for detection of a bleeding ulcer and/or gastrointestinal bleeding.

The methods and devices described herein can be used in procedures to identify and to direct biopsy of gastrointestinal tumors. The nIR imaging techniques described herein can be used to identify tumors located near the epithelial surface, for example, the duodenal wall. For example, by injecting a patient with a dye which is selectively absorbed by the vasculature of a tumor and the tumor is located within a distance from the epithelial surface such that nIR light emitted from the endoscope reaches the absorbed dye and light fluoresced from the dye can be detected by the imaging system. Such nIR imaging techniques can similarly be used in detecting pancreatic masses, for example, after accessing the pancreatic duct or common bile duct.

While the foregoing systems and methods have been described in some detail for purposes of clarity and understanding, it will be appreciated by one skilled in the art from a reading of this disclosure that various changes in form and detail can be made without departing from the true scope of the invention and appended claims.

What is claimed is:

1. An imaging system for an endoscope comprising:
a light source capable of emitting near-infrared (nIR) light;
an objective lens;
an optical filter;
an imaging sensor; and
an image display unit.

2. The imaging system of claim 1 wherein the light source is capable of emitting nIR light having a wavelength of 800 nm.

3. The imaging system of claim 1 wherein the optical filter is a band-pass filter centered at 830 nm or a long-pass filter excluding light below 810 to 820 nm.

4. The imaging system of claim 1 wherein the light source is further capable of emitting visible light.

5. The imaging system of claim 1 wherein the imaging sensor is a camera.

6. The imaging system of claim 5 wherein the camera is a solid state nIR sensitive camera.

7. The imaging system of claim 1 wherein the image display unit is a monitor.

8. The imaging system of claim 1 wherein said endoscope is adapted for use in endoscopic retrograde cholangiopancreatography (ERCP).

9. The imaging system of claim 1 wherein said endoscope is adapted for use in esophagogastroduodenoscopy (EGD).

10. An endoscope comprising:
a distal portion for insertion into a patient;
a proximal portion which is external to the patient; and
an imaging system having a light source capable of emitting near-infrared (nIR) light; an objective lens; an optical filter; and an imaging sensor.

11. The endoscope of claim 10 wherein the light source and objective lens are positioned at a distal end of the endoscope.

12. The endoscope of claim 10 wherein the optical filter and imaging sensor are positioned at a proximal end of the endoscope.

13. The endoscope of claim 10 wherein the light source is capable of emitting nIR light having a wavelength of 800 nm.

14. The endoscope of claim 10 wherein the optical filter is a band-pass filter centered at 830 nm or a long-pass filter excluding light below 810 to 820 nm.

15. The endoscope of claim 10 wherein the light source is further capable of emitting visible light.

16. The endoscope of claim 10 wherein the imaging sensor is a camera.

17. The endoscope of claim 16 wherein the camera is a solid state nIR sensitive camera.

18. The endoscope of claim 10 further comprising at least one lumen.

19. The endoscope of claim 18 further comprising at least one medical device inserted in said at least one lumen.

20. The endoscope of claim 19 wherein the at least one medical device is a catheter or a guide wire.

21. The endoscope of claim 10 wherein the endoscope is adapted for use in endoscopic retrograde cholangiopancreatography (ERCP).

22. The endoscope of claim 10 wherein the endoscope is adapted for use in esophagogastroduodenoscopy (EGD).

23. An endoscopic retrograde cholangiopancreatography (ERCP) method comprising:
injecting a patient with a dye which emits near-infrared (nIR) light upon excitation and which is absorbed into biliary epithelium;
positioning, in the patient's duodenum, an endoscope capable of emitting and detecting near-infrared light;
emitting near-infrared light from the endoscope;
detecting near-infrared light emitted by the dye to provide an image for guidance of the endoscope for cannulation of intraduodenal portion of the bile duct; and
cannulating the intraduodenal portion of the bile duct.

24. The method of claim 23 wherein the dye is indocyanine green.

25. The method of claim 23 wherein the endoscope comprises:
a distal portion for insertion into a patient;
a proximal portion which is external to the patient; and
an imaging system having a light source capable of emitting near-infrared light; an objective lens; an optical filter; and an imaging sensor.

26. The method of claim 25 wherein the light source and objective lens are positioned at a distal end of the endoscope.

27. The method of claim 25 wherein the optical filter and imaging sensor are positioned at a proximal end of the endoscope.

28. The method of claim 25 wherein the light source emits nIR light having a wavelength of 800 nm.

29. The method of claim 25 wherein the optical filter is a band-pass filter centered at 830 nm or a long-pass filter excluding light below 810 to 820 nm.

30. The method of claim 25 wherein the light source also emits visible light.

31. The method of claim 25 wherein the imaging sensor is a camera.

32. The method of claim 31 wherein the camera is a solid state nIR sensitive camera.

33. The method of claim 25 wherein the endoscope further comprises at least one lumen and at least one medical device inserted in said at least one lumen.

34. The method of claim 33 wherein the at least one medical device is a catheter or a guide wire.
